(19) United States
(12) Patent Application Publication
(10) Pub. No.: US 2024/0042166 A1
(43) Pub. Date: Feb. 8, 2024

(54) SEMICONDUCTOR LIGHT-EMITTING DEVICE

(71) Applicant: TIANJIN SANAN OPTOELECTRONICS CO., LTD., Tianjin (CN)

(72) Inventors: Weifan KE, Tianjin (CN); Chun-Yi WU, Tianjin (CN); Bing-xian CHUNG, Tianjin (CN)

(21) Appl. No.: 18/491,089

(22) Filed: Oct. 20, 2023

Related U.S. Application Data
(62) Division of application No. 16/229,190, filed on Dec. 21, 2018, now Pat. No. 11,826,515.
Application No. 16/229,190 is a division of application No. 13/984,024, filed on Nov. 22, 2013, now Pat. No. 10,195,393.
(60) Provisional application No. 61/441,258, filed on Feb. 9, 2011.

(51) Int. Cl. A61M 25/00; A61M 5/158; B29C 45/00; A61M 25/06

(52) U.S. Cl. CPC A61M 25/001; A61M 5/158; A61M 25/0009; B29C 45/00; A61M 25/0606; A61M 25/0097; B29C 45/1615

(57) ABSTRACT

A semiconductor light-emitting device includes a bonding substrate, a multi-layered metal unit, and a semiconductor lighting unit. The bonding substrate includes an upper surface and a lower surface opposite to the upper surface. The multi-layered metal unit is disposed on the upper surface of the bonding substrate such that an exposed region of the upper surface of the bonding substrate is exposed from the multi-layered metal unit. The semiconductor lighting unit is disposed on the multi-layered metal unit opposite to the bonding substrate. A method for manufacturing the semiconductor light-emitting device is also disclosed.

### CROSS-REFERENCE TO RELATED APPLICATION

This application is a divisional application of U.S. patent application Ser. No. 17/157,127, which is a bypass continuation-in-part application of International Application No. PCT/CN2018/097578 filed on Jul. 27, 2018. The entire content of the international patent application is incorporated herein by reference.

### FIELD

The disclosure relates to a light-emitting device, and more particularly to a semiconductor light-emitting device with improved light-emitting efficiency and reliability.

### BACKGROUND

In order to obtain a light-emitting diode (LED) chip with a high brightness, a high power or a high heat radiation rate, a conventional LED epitaxial structure grown on a growth substrate is transferred to a transferring substrate that includes a metal reflection layer or a metal bonding layer, and the growth substrate is then removed by a chemical wet etching process or a laser lift-off (LLO) process. Next, the transferred LED epitaxial structure disposed on the metal reflection layer or the metal bonding layer is partially etched to form a cutting channel. Afterwards, such LED epitaxial structure is cut along the cutting channel using a dicing saw or a laser beam, so as to obtain a plurality of the LED chips.

However, since use of the dicing saw might enlarge an area of the cutting channel, the resultant LED chips might be susceptible to damage (e.g., collapse). Application of the laser beam for cutting the LED epitaxial structure seems to be more promising than that of the dicing saw since the laser beam allows the separated LED chips to have a flat breaking surface and a relatively narrow cutting channel. However, a large amount of burnt metal impurities would be generated when the laser beam focuses on the metal reflection layer or the metal bonding layer during the cutting process. Such burnt metal impurities would sputter on a sidewall of an light-emitting layer of the LED chips, resulting in an electrical leakage of the light-emitting layer and a decreased brightness due to light emitted from the light-emitting layer being absorbed by such burnt metal impurities.

### SUMMARY

An object of the disclosure is to provide a semiconductor light-emitting device that can alleviate at least one of the drawbacks of the prior art.

According to the disclosure, the semiconductor light-emitting device includes a bonding substrate, a multi-layered metal unit, and a semiconductor lighting unit.

The bonding substrate includes an upper surface and a lower surface opposite to the upper surface.

The multi-layered metal unit is disposed on the upper surface of the bonding substrate such that an exposed region of the upper surface of the bonding substrate is exposed from the multi-layered metal unit.

The semiconductor lighting unit is disposed on the multi-layered metal unit opposite to the bonding substrate.

Another object of the disclosure is to provide a method for manufacturing at least one semiconductor light-emitting device that can alleviate or eliminate at least one of the drawbacks of the prior art.

According to the disclosure, the method includes the following steps (a) to (d).

In step (a), a semiconductor light-emitting structure is provided and includes a bonding substrate, a multi-layered metal unit, and a semiconductor lighting unit. The bonding substrate has an upper surface and a lower surface opposite to the upper surface. The multi-layered metal unit is disposed on the upper surface of the bonding substrate. The semiconductor lighting unit is disposed on the multi-layered metal unit opposite to the bonding substrate.

In step (b), a portion of the semiconductor lighting unit is removed to form a first recess structure on the multi-layered metal unit.

In step (c), a portion of the multi-layered metal unit is removed along the first recess structure to form a second recess structure that extends through the multi-layered metal unit to expose an exposed region of the bonding substrate.

In step (d), the bonding substrate is diced along the exposed region of the bonding substrate, so as to obtain the semiconductor light-emitting device from the semiconductor light-emitting structure.

### BRIEF DESCRIPTION OF THE DRAWINGS

Other features and advantages of the disclosure will become apparent in the following detailed description of the embodiments with reference to the accompanying drawings, of which:

FIG. 1 is a schematic view illustrating an embodiment of a semiconductor light-emitting device according to the disclosure;

FIG. 2 is a schematic side view illustrating the embodiment of the semiconductor light-emitting device according to the disclosure; and

FIGS. 3 to 6, 7a and 7b show schematic side views illustrating consecutive steps of a method for manufacturing a second embodiment of the semiconductor light-emitting device according to the disclosure, in which FIG. 6 shows a variation of FIG. 5, and FIGS. 7a and 7b show a plurality of explosion points formed in step S4 of the method.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

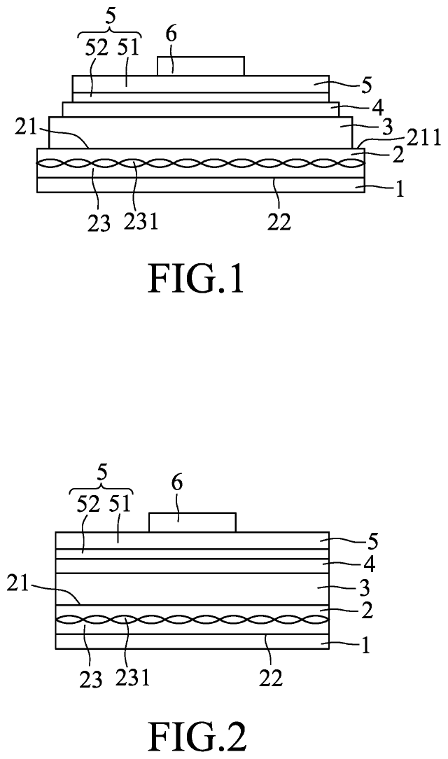

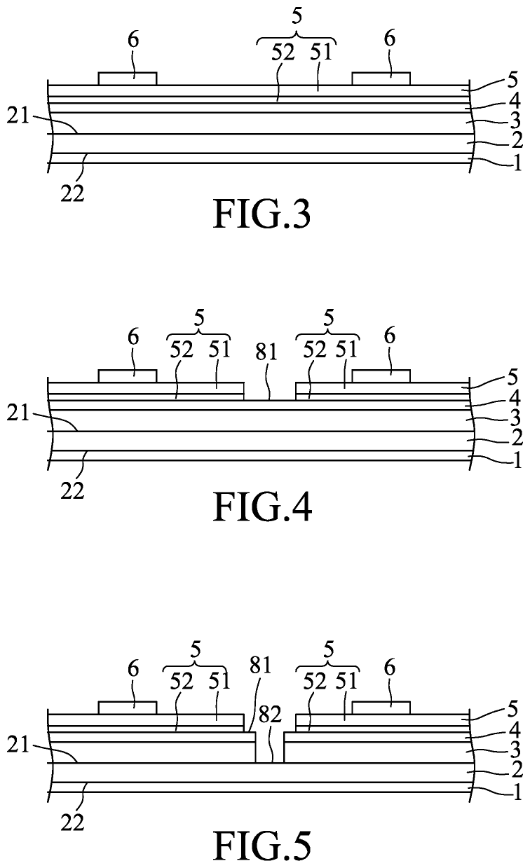

Referring to FIGS. 1 and 2, a first embodiment of a semiconductor light-emitting device according to the present disclosure includes a bonding substrate 2, a multi-layered metal unit 3, and a semiconductor lighting unit 5.

The bonding substrate 2 includes an upper surface 21, a lower surface 22 opposite to the upper surface 21, and a side surface 23 interconnecting the upper surface 21 and the lower surface 22. A portion of the side surface 23 may be formed with a concave-convex structure 231 which may be formed from continuous or discontinuous explosion points generated by a laser cutting process as described below. In certain embodiments, the concave-convex structure 231 is located at a position that is relatively near one of the upper surface 21 and the lower surface 22 of the bonding substrate 2, or at a center region of the side surface 23 of the bonding substrate 2. In other embodiments, the concave-convex structure 231 extends to one of the upper surface 21 and the lower surface 22 of the bonding substrate 2. A distance from the upper surface 21 to the concave-convex structure 231 may be one third to half of a distance from the upper surface 21 to the lower surface 22. The concave-convex structure 231 may have a roughness greater than that of the remaining region of the side surface 23 of the bonding substrate 2. The bonding substrate 2 may be made of a non-metallic material, e.g., a semiconductor material. In certain embodiments, the bonding substrate 2 is an electrically conductive substrate that is configured to absorb a laser radiation during the laser cutting process. Examples of the electrically conductive substrate may include, but are not limited to, a nitride-based substrate, a silicon (Si) substrate (e.g., p-type silicon or n-type silicon substrate), and a silicon carbide (SiC) substrate.

The multi-layered metal unit 3 is disposed on the upper surface 21 of the bonding substrate 2 such that an exposed region 211 of the upper surface 21 of the bonding substrate 2 is exposed from the multi-layered metal unit 3. That is, the multi-layered metal unit 3 and the bonding substrate 2 cooperate to form a stage structure (i.e., the exposed region 211). The exposed region 211 of the upper surface 21 may have a width ranging from 2 μm to 10 μm, such as from 3 μm to 6 μm. The multi-layered metal unit 3 may include one of a bonding layer, a metal reflection layer, an ohmic contact layer, a blocking layer, and combinations thereof.

The bonding layer is disposed on the bonding substrate 2 for bonding the multi-layered metal unit 3 to the bonding substrate 2. The bonding layer may be made of a metallic material (such as Au) that can electrically and mechanically connect to the bonding substrate 2.

The ohmic contact layer is configured to form ohmic contact with the bonding substrate 2. The ohmic contact layer may be made of a metallic material such as Au, Ti, and Al.

The metal reflection layer is configured to reflect a light emitted from the semiconductor lighting unit 5 back thereto, and may include a conductive material with a high reflective index (e.g., at least 80%) to the light. For example, the metal reflection layer may include a metallic material such as a metal (e.g., Al, Au, or Ag) and a metal alloy thereof. In certain embodiments, the metal reflection layer is made of Au and has a predetermined thickness. The metal reflection layer and the ohmic contact layer may be made of an identical material.

The blocking layer is configured to prevent diffusion of metal atoms of the metal reflection layer into the semiconductor lighting unit 5. The blocking layer may be made of a metallic material (e.g., Ti or Pt).

The semiconductor lighting unit 5 is disposed on the multi-layered metal unit 3 opposite to the bonding substrate 2. In certain embodiments, the semiconductor lighting unit 5 is disposed on a portion of the multi-layered metal unit 3, such that an exposed portion of the multi-layered metal unit 3 is exposed from the semiconductor lighting unit 5. That is, the multi-layered metal unit 3 and the semiconductor lighting unit 5 cooperate to form another stage structure (i.e., the exposed portion of the multi-layered metal unit 3) which may have a width ranging from 1.5 μm to 10 μm, such as from 3 μm to 8 μm. An area of a projection of the semiconductor lighting unit 5 on the bonding substrate 2 may be at least 50% (such as at least 70%, at least 80%, etc.) of an area of the upper surface 21 of the bonding substrate 2.

The semiconductor lighting unit 5 may include a first-type contact layer and a second-type contact layer (not shown in the figures), and a light-emitting element 51 disposed between the first-type contact layer and the second-type contact layer. The term “first-type” refers to being doped with a first type dopant, and the term “second-type” refers to being doped with a second type dopant that is opposite in conductivity to the first type dopant. For instance, the first type dopant may be a p-type dopant, and the second type dopant may be an n-type dopant, and vice versa.

Each of the light-emitting element 51, the first-type contact layer and the second-type contact layer may be made of a group III-V semiconductor material such as a binary semiconductor material (e.g., gallium arsenide (GaAs)-based material, gallium phosphide (GaP)-based material, or indium phosphide (InP)-based material), a ternary semiconductor material (e.g., indium gallium arsenide (InGaAs)-based material, indium gallium phosphide (InGaP)-based material, or aluminium gallium arsenide (AlGaAs)-based material), and a quaternary semiconductor compound (e.g., aluminium gallium indium phosphide (AlGaInP)-based material). The light-emitting element 51 may include a p-type cladding layer made of p-type AlGaInP-based material, an n-type cladding layer made of n-type AlGaInP-based material, and an active layer that is disposed between the p-type and n-type cladding layers, that is configured to emit a light having a predetermined wavelength, and that is made of an undoped AlGaInP-based material. The semiconductor lighting unit 5 may be first grown on a growth substrate made of a gallium arsenide (GaAs)-based material, and is then transferred to the bonding substrate 2.

The semiconductor lighting unit 5 may further include a current spreading layer 52 disposed between the light-emitting element 51 and the multi-layered metal unit 3. The current spreading layer 51 may be made of p-type GaP, and is adapted for spreading current around the p-type cladding layer. In certain embodiments, the light-emitting element 51 may be disposed on a portion of the current spreading layer 52, such that an exposed portion of the current spreading layer 52 is exposed from the light-emitting element 51, i.e., forming a stage.

The second-type contact layer is formed on a light extraction surface of the light-emitting element 51, i.e., a surface of the n-type cladding layer that is opposite to the active layer. In certain embodiments, in order to increase the light emitting efficiency of the semiconductor light-emitting device, two opposite surfaces of the light-emitting element 51 and/or a side surface of the light-emitting element 51 are formed with a concave-convex portion. The concave-convex portion may be covered with a transparent insulating film.

The semiconductor light-emitting device may further include a transparent insulating layer 4 that is disposed between the semiconductor lighting unit and the multi-layered metal unit 3, and that may be formed as one of a single layer structure and a multi-layered structure.

The semiconductor light-emitting device may further include, between the first-type contact layer and the metal reflection layer of the multi-layered metal unit 3, a dielectric layer (i.e., an insulating film), and an ohmic contact portion that is disposed on a region free of the dielectric layer and that is configured to electrically connect the first-type contact layer to the metal reflection layer.

Specifically, the dielectric layer is formed with a through hole that is defined by a hole-defining wall and that extends from the first-type contact layer and the metal reflection layer. The ohmic contact portion is formed on the hole-defining wall to electrically connect the first-type contact layer to the metal reflection layer. The ohmic contact portion may be made of a metal (such as Au and Zn), or a metal alloy (e.g., AuZn alloy).

The dielectric layer may be formed as a single layer structure, i.e., an insulating film made of silicon dioxide (SiO₂) or silicon nitride (Si₃N₄). Alternatively, the dielectric layer may be formed as a multi-layered insulating structure, which may include multiple insulating films having different refractive indices. For example, the insulating films of the multi-layered insulating structure may have refractive indices that gradually decrease in a direction away from the light extraction surface of the light-emitting element 51 and/or the side surface of the light-emitting element 51. Alternatively, the multi-layered insulating structure may include multiple pairs of insulating films, each pair containing a first insulating film (such as silicon dioxide (SiO₂) film) and a second insulating film (such as silicon nitride (Si₃N₄) film) having a refractive index different from that of the first insulating film. The first insulating films and the second insulating films in the multi-layered insulating structure may be alternately stacked. In certain embodiments, the multi-layered insulating structure is a distributed bragg reflector (DBR) structure that includes multiple pairs of films, each pair containing a SiO₂ film having a predetermined thickness and a titanium oxide (TiO₂) film having a predetermined thickness, and the SiO₂ films and the TiO₂ films are alternately stacked.

The semiconductor light-emitting device further includes a front metal electrode 6 and a conductive metal layer 1 (or a backside metal electrode).

The front metal electrode 6 is disposed on the second-type contact layer of the semiconductor lighting unit 5 opposite to the light-emitting element 51, and is adapted to be bonded to a pad electrode of a pad through a wire. There are no particular limitations on the shape of the front metal electrode 6. For example, the front metal electrode 6 may be in a circle shape (see FIG. 7b) or in a polygonal shape (e.g., hexagon). The front metal electrode 6 is made of a metallic material (e.g., Au, Ge, or Ni) so as to form an ohmic contact with the n-type contact layer. The pad electrode that is in contact with a surface of the front metal electrode 6 may be made of a metallic material such as Ti and Au.

The conductive metal layer 1 is disposed on the lower surface 22 of the bonding substrate 2, and is electrically connected to the bonding substrate 2. The conductive metal layer 1 may be made of a metallic material such as Ti and Au. In this embodiment, the conductive metal layer 1 is made of Au.

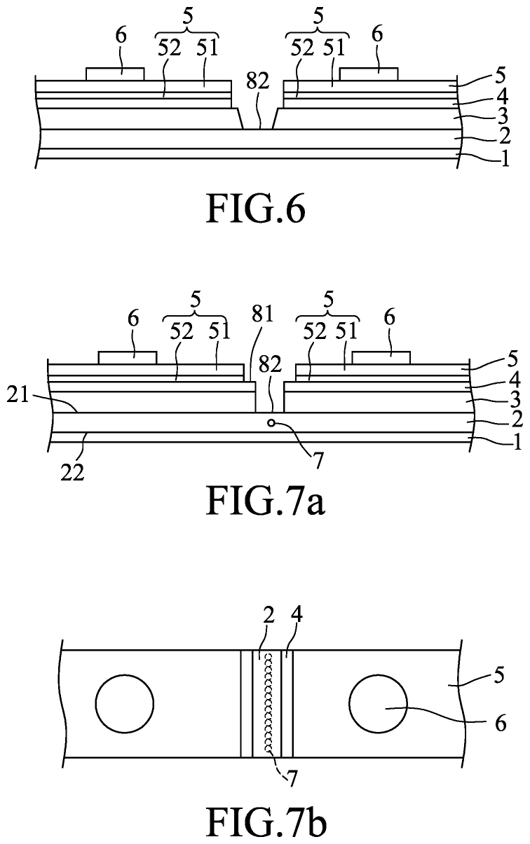

Referring to FIGS. 3 to 7b, a method for manufacturing at least one of the semiconductor light-emitting device according to a second embodiment of this disclosure includes the following consecutive steps S1 to S4. The second embodiment is generally similar to the first embodiment, except that in the second embodiment, an area of a projection of the transparent insulating layer 4 on the bonding substrate 2 is the same as an area of a projection of the multi-layered metal unit 3 on the bonding substrate 2 (see FIG. 7a).

In step S1, a semiconductor light-emitting structure as shown in FIG. 3 is provided. The semiconductor light-emitting structure includes the conductive metal layer 1, and the bonding substrate 2, the multi-layered metal unit 3, the transparent insulating layer 4, the semiconductor lighting unit and at least one front metal electrode 6 that are sequentially disposed on the conductive metal layer 1.

In step S2, as shown in FIG. 4, a portion of the semiconductor lighting unit 5 is removed to form a first recess structure 81 on the multi-layered metal unit 3.

To be specific, the semiconductor lighting unit is subjected to a photolithography process which includes application of a photoresist layer on a surface of the semiconductor lighting unit 5 opposite to the bonding substrate 2, light-exposure and development, etching treatment, and removal of the photoresist layer. The first recess structure 81 may extend through the semiconductor lighting unit 5, and terminate at and expose the transparent insulating layer 4. The etching treatment may be a dry etching process.

In step S3, as shown in FIG. 5, a portion of the multi-layered metal unit 3 is removed along the first recess structure 81 by, e.g. a photolithography process, to form a second recess structure 82 that extends through the multi-layered metal unit 3 so as to expose an exposed region of the bonding substrate 2. The second recess structure 82 has a width that is greater than a width of the first recess structure 81.

Specifically, the semiconductor lighting unit 5 (including a side wall of the first recess structure 81) and a peripheral region of a bottom wall of the first recess structure 81 (i.e., a portion of the exposed transparent insulating layer 4) are covered by a photoresist layer, so as to prevent the semiconductor lighting unit 5 from being etched and to avoid the loss of the light-emitting area in subsequent etching treatment. Then, the remaining portion of the first recess structure 81 (i.e., the uncovered portion of the transparent insulating layer 4) and the multi-layered metal unit 3 are subjected to an etching treatment to expose the bonding substrate 2. The etching treatment may include a dry etching process and/or a wet etching process depending on the materials to be removed. For example, the transparent insulating layer 4 and the current spreading layer 52, if present, may be removed by a dry etching process. The multi-layered metal unit 3 may be removed by a wet etching process and a dry etching process. For instance, the metal reflection layer made of AuZn or Au is removed by the wet etching process, and the blocking layer made of Ti or Pt is removed by the dry etching process. By virtue of the etching treatment in this step which involves several etching processes as mentioned above, at least one of the multi-layered metal unit 3, the transparent insulating layer 4 and the current spreading layer 52 (if present) may have an area that gradually changes (e.g., increase in size) in a direction towards the bonding substrate 2. In one form, the multi-layered metal unit 3 is formed with an inclined side surface and has an area that gradually increases in a direction towards the bonding substrate 2, and a projection of the transparent insulating layer 4 on the bonding substrate 2 is smaller than that of the multi-layered metal unit 3 (see FIG. 6). With such structure, light emitted from a side surface of the semiconductor lighting unit 5 is capable of being reflected by the multi-layered metal unit 3.

In step S4, the bonding substrate 2 is diced along the exposed region of the bonding substrate 2, so as to obtain the semiconductor light-emitting device from the semiconductor light-emitting structure.

In this embodiment, step S4 is implemented by a laser stealth dicing process. Specifically, as shown in FIGS. 7a and 7b, the bonding substrate 2 is first formed with a plurality of explosion points 7 corresponding in position to the exposed region of the upper surface 21 by focusing a laser beam inside the bonding substrate 2. By virtue of adjusting the power of the laser beam, a distance from the upper surface 21 of the bonding substrate 2 to the explosion points 7 may be one third to half of a distance from the upper surface 21 to the lower surface 22 of the bonding substrate 2. Next, the bonding substrate 2 of the semiconductor light-emitting structure is cut along the exposed region of the bonding substrate 2 to expose the explosion points 7, so as to obtain the semiconductor light-emitting device from the semiconductor light-emitting structure. Since the explosion points 7 have decreased stress, the bonding substrate 2 may be formed with the concave-convex structure 231 at the side surface 23 which corresponds in position to the explosion points 7.

In a variation of this embodiment, step S4 is implemented by a laser scribing and breaking process, so as to reduce the area to be cut, thereby increasing an area of the light-emitting region and the light-emitting efficiency of the thus obtained semiconductor light-emitting device. Specifically, the exposed region of the bonding substrate 2 is first scribed using laser to form a recess that has a predetermined depth in the bonding substrate 2. Next, the bonding substrate 2 is subjected to breaking using a saw along the recess, so as to obtain the semiconductor light-emitting device from the semiconductor light-emitting structure.

In summary, by virtue of forming the second recess structure 82 in the multi-layered metal unit 3 to expose the exposed region 211 of the upper surface 21 of the bonding substrate 2 therefrom, during the dicing step, the laser beam can be prevented from directly being focused on the multi-layered metal unit 3, so as to avoid generation of burnt metal impurities that may sputter on the sidewall of the semiconductor lighting unit 5. Therefore, electrical leakage of the semiconductor light-emitting device of this disclosure can be greatly reduced, so that light-emitting efficiency and stability of the semiconductor light-emitting device can be improved.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A method for manufacturing at least one semiconductor light-emitting device, comprising the steps of:
(a) providing a semiconductor light-emitting structure that includes
a bonding substrate having an upper surface and a lower surface opposite to the upper surface;
a multi-layered metal unit disposed on the upper surface of the bonding substrate; and
a semiconductor lighting unit disposed on the multi-layered metal unit opposite to the bonding substrate;
(b) removing a portion of the semiconductor lighting unit to form a first recess structure on the multi-layered metal unit;
(c) removing a portion of the multi-layered metal unit along the first recess structure to form a second recess structure that extends through the multi-layered metal unit to expose an exposed region of the bonding substrate; and
(d) dicing the bonding substrate along the exposed region of the bonding substrate, so as to obtain the semiconductor light-emitting device from the semiconductor light-emitting structure.

2. The method of claim 1, wherein:
in step (a), the semiconductor light-emitting structure further includes a transparent insulating layer disposed between the multi-layered metal unit and the semiconductor lighting unit; and
in step (c), a portion of the transparent insulating layer is removed, the second recess structure extending through said transparent insulating layer.

3. The method of claim 1, wherein step (d) is implemented by one of a laser scribing and breaking process and a laser stealth dicing process.

4. The method of claim 2, wherein the transparent insulating layer is formed as one of a single layer structure and a multi-layered structure, and in step (c), the transparent insulating layer is removed by a dry etching process.

5. The method of claim 2, wherein, in step (c), the portion of the exposed multi-layered metal unit is removed by a wet etching process and a dry etching process.
